(19) United States
(12) Patent Application Publication
(10) Pub. No.: US 2013/0115697 A1
(43) Pub. Date: May 9, 2013

(54) METHODS AND APPARATUS FOR ENHANCED RECOVERY OF CELLS FROM TISSUE SAMPLES

(71) Applicants: Eckhard U. Alt, Houston, TX (US); Michael E. Coleman, Houston, TX (US); Ron Stubbers, Houston, TX (US)

(72) Inventors: Eckhard U. Alt, Houston, TX (US); Michael E. Coleman, Houston, TX (US); Ron Stubbers, Houston, TX (US)

(21) Appl. No.: 13/385,599

(22) Filed: Nov. 3, 2011

(51) Int. Cl. C12N 5/071

(52) U.S. Cl. CPC C12N 5/0667
USPC 435/379

(57) ABSTRACT

This document describes methods and an apparatus for recovery of cells from a tissue sample. In some embodiments, at least two rounds of acceleration and deceleration are performed.

### TECHNICAL FIELD

This invention relates to methods and an apparatus for recovery of cells, and more particularly to recovery of cells from a tissue sample using two or more acceleration and deceleration steps under centrifugal force.

### BACKGROUND

Autologous grafting of tissue harvested by lipoaspiration is a common procedure in cosmetic surgery for both small (e.g., nasolabial folds) and large (buttocks or breast) volume filling applications. The primary benefits of this procedure termed “autologous fat grafting” are lower cost versus synthetic fillers and no immune rejection since the patient's own tissue is used. Currently, multiple methods of lipoaspirate collection and processing are employed to obtain tissue for grafting. Factors that determine clinical outcomes following autologous fat grafting have not been fully elucidated. However, it is widely recognized that improving the persistence of the graft is an area of significant need.

One potential method to improve persistence of the autologous graft would be to augment the graft tissue with progenitor cells or otherwise process the lipoaspirate to yield graft tissue with the highest potential for persistence after grafting. A population of non-lipid filled cells, referred to as stromal vascular cells (SVC), have been extracted from lipoaspirate using collagenase digestion followed by filtration and centrifugation. Combining cells prepared by this methodology with lipoaspirate leads to enhanced persistence of the graft in animal models and is currently in clinical use in countries where permissible under government regulations. This approach is limited by several factors including, but not limited to, the cost of high quality enzyme to digest tissue matrix, time and number of steps involved in tissue processing, and regulatory acceptance. A method that employed purely physical means of recovering SVC from adipose tissue or enriching SVC in processed lipoaspirate would provide significant advantages.

### SUMMARY

This document is based on the discovery of methods and apparatus for enhancing the recovery of cells from human or animal tissue. The methods described herein are easy to perform in a short period of time.

In one embodiment, this document features a method for recovering a regenerative platform from a tissue sample (e.g., lipoaspirate, adipose tissue, and combinations thereof). The method includes providing a tissue sample housed in a first tissue collection container adapted for an automated tissue processing unit, wherein the automated tissue processing unit comprises a removable rotating apparatus comprising at least two cavities, wherein each cavity is configured for detachably inserting a tissue collection container within the cavity wherein the tissue sample comprises a suspension of tissue pieces in an aqueous fluid; and subjecting the tissue sample to at least one round of centrifugation of at least 400×g for at least about 5 minutes using the automated tissue processing unit, thereby separating a tissue sample concentrate from the tissue sample, wherein the tissue sample concentrate comprises a regenerative platform. The method further can include extruding the tissue sample through an orifice prior to placing the tissue sample into the automated tissue processing unit. The tissue sample concentrate can have a higher concentration of the regenerative platform compared to an otherwise corresponding method absent the extruding the tissue sample through an orifice. The method further can include transferring the tissue sample concentrate from the first tissue collection container into a second collection container by a closed system method. In some embodiments, at least one protease can be added to the second collection container. The tissue sample concentrate can be subjected to at least two rounds of acceleration, wherein each round of acceleration is followed by a round of deceleration, thereby disaggregating the tissue sample concentrate. In some embodiments, the tissue sample concentrate can be filtered to obtain an injectable regenerative platform.

In any of the methods described herein, the method further can include administering at least a portion of the injectable regenerative platform into a subject at an injection site, whereby the injection alters an area at or near the injection site.

This document also features a method for disaggregating a tissue sample concentrate having a regenerative platform therein, wherein the method comprises providing a tissue sample concentrate housed in a second tissue collection container adapted for an automated tissue processing unit, wherein the tissue collection container comprises at least one protease; and subjecting the tissue sample concentrate to at least two rounds of acceleration, wherein each round of acceleration is followed by a round of deceleration, and wherein at least two rounds of acceleration and deceleration are performed at a rate of at least 10×g thereby disaggregating the tissue sample concentrate. The method further can include filtering the disaggregated tissue sample concentrate to obtain an injectable regenerative platform.

This document also features a removable rotating apparatus comprising at least two cavities, wherein each cavity is configured for detachably inserting a tissue collection container within the cavity, wherein the removable rotating apparatus is configured to rotate within an automated tissue processing unit for separating a tissue sample concentrate from a tissue sample. The removable rotating apparatus can include a radio-frequency identification (RFID) tag attached thereto that allows the removable rotating apparatus to be identified by the automated tissue processing unit. The removable rotating apparatus can include autoclavable materials.

In another aspect, this document features an automated tissue processing unit for isolating a tissue sample concentrate from a tissue sample. The automated tissue processing unit can include a removable rotating apparatus comprising at least two cavities, wherein each cavity is configured for detachably inserting a tissue collection container within the cavity. The automated tissue processing unit can include a temperature control device. The automated tissue processing unit can be configured to have at least two stop-start intervals of acceleration. The removable rotating apparatus can have at least one pre-determined specification that allows the automated tissue processing unit to identify the removable rotating apparatus.

In another aspect, a modified centrifuge is provided that can be used to perform at least two series of rapid acceleration and deceleration steps under centrifugal force. Such steps can be performed in a thermally regulated environment (e.g., 35-42° C.) in the presence of one or more enzymes (e.g., a collagenase and a neutral protease) to enhance the degradation of the extracellular matrix and release of cells. Centrifugation can be used to recover cells released from the extracellular matrix. The methods and apparatus described herein can be used to process any human or animal tissue that contains blood vessels. The methods and apparatus are particularly useful for recovering cells from adipose tissue (e.g., subcutaneous or intra-abdominal adipose tissue), which is rich in vascularization and easy to recover from a subject.

This document also provides a method for recovering cells from tissue. The method includes providing a tissue sample housed in a container adapted for a centrifuge, the tissue sample including a suspension of tissue pieces in an aqueous fluid; and subjecting the tissue sample to a plurality of acceleration and deceleration steps using centrifugal force. The tissue sample can include human tissue or animal tissue, and can contain blood vessels. The tissue sample can be adipose tissue such as lipoaspirate. The method can include maintaining a temperature of from 26° C. to 42° C. inside the container while subjecting the tissue sample to the plurality of acceleration and deceleration steps. The tissue sample can be subjected to the plurality of acceleration and deceleration steps in the presence of one or more enzymes (e.g., a collagenase, other protease, or a mixture thereof).

In some embodiments, each of the acceleration steps can be performed for 5 to 20 seconds and each of the deceleration steps can be performed for 3 to 20 seconds. The tissue sample can be subjected to the plurality of acceleration and deceleration steps for 5 minutes to 180 minutes (e.g., 20 minutes to 60 minutes). In one embodiment, the tissue sample is subjected to at least three cycles of acceleration to 200×g and deceleration to 1×g per minute for 30 minutes.

In another aspect, this document features a method for recovering cells from tissue. The method includes providing a tissue sample housed in a container adapted for a centrifuge, the tissue sample including a suspension of tissue pieces in an aqueous fluid; subjecting the sample to a plurality of acceleration and deceleration steps using centrifugal force applied through a rotating element, wherein the rotating element comprises a shaft and one or more arms that extend from the shaft, wherein (i) the one or more arms are supported from the shaft in such a manner that when the shaft rotates, the one or more arms swing upward and outward relative to the shaft or (ii) the one or more arms are supported at a fixed angle, wherein the containers attached to the arms are held in such a position that gravitational force on material is opposite of applied centrifugal force, wherein the applied centrifugal force ranges from about 50 g to about 4000 g. Each of the acceleration steps can be performed for 5 to 20 seconds. Each of the deceleration steps can be performed for 3 to 20 seconds. The tissue sample can be subjected to the plurality of acceleration and deceleration steps for 5 minutes to 180 minutes (e.g., 20 minutes to 60 minutes). In one embodiment, the tissue sample is subjected to at least three cycles of acceleration to 200×g and deceleration to 1×g per minute for 30 minutes.

This document also features a method for recovering regenerative cells from tissue. The method includes providing a tissue sample housed in a container adapted for a centrifuge, the tissue sample comprising a suspension of tissue pieces in an aqueous fluid; subjecting the sample to a plurality of acceleration and deceleration steps using centrifugal force; and centrifuging the sample at 400 to 4000×g to isolate cellular components. The sample, when subjected to centrifugation at 400 to 4000×g, can be housed within a container that includes an elongated cylindrical central portion; a first end portion integrally formed with the central portion; and a second open end portion integrally formed with the central portion, wherein the first end portion narrows down to a narrow opening, and comprises a collection portion protruding from the end portion at the narrow opening, wherein the collection portion is capable of receiving and storing a liquid and comprises a removable plug to seal the first end portion from the collection portion. In any of the methods described herein, the container can include a porous insert, wherein the porous insert is composed of a biocompatible material and having a pore size ranging from 0.5 mm to 5 mm, wherein the porous insert enhances the dissociation of cells from the extracellular matrix of the tissue sample when the tissue sample is subjected to said plurality of acceleration and deceleration steps. The porous insert can be substantially cylindrical in shape, an inverted substantially conical shape, or can bisect the container into upper and lower portions.

In any of the methods described herein, the container can include a plurality of particles, wherein the particles are at least 100 micrometer in diameter and composed of one or more biocompatible materials, wherein the particles enhance the dissociation of cells from the extracellular matrix of the tissue sample when the tissue sample is subjected to the plurality of acceleration and deceleration steps. The plurality of particles can include particles of different specific gravities or shapes.

In any of the methods described herein, the container can include a shaft disposed vertically in the internal lumen of the container, the shaft further including a plurality of arms disposed along a length of the shaft and extending substantially radially from the shaft into the lumen of the container, wherein the arms enhance the dissociation of cells from the extracellular matrix of the tissue sample when the tissue sample is subjected to the plurality of acceleration and deceleration steps. The arms can be of different shapes or sizes. The container can include a removable lid, wherein the shaft is affixed to the removable lid. The shaft can be rotatably affixed to the container. The shaft can be moveable within the lumen of the container.

In another aspect, this document features a container or container assembly that includes an elongated cylindrical central portion; a first end portion integrally formed with the central portion; a second open end portion integrally formed with the central portion; and a port extending radially outward from the elongated cylindrical portion, wherein the first end portion narrows down to a narrow opening, and includes a collection portion protruding from the end portion at the narrow opening, wherein the collection portion is capable of receiving and storing a fluid and comprises a removable plug to seal the first end portion from the collection portion. The removable plug can allow fluid to flow from the end portion into the collection portion upon centrifugal force, pressure, digestion with an enzyme, or physical removal. The collection portion can be detachable from the first end portion. The collection portion can include an aqueous fluid. The second open end includes a mating portion.

This document also features a container assembly that includes a first container; a second container; and a coupling device adapted to couple the first container to the second container. The first container is described above. The second container includes an elongated cylindrical central portion, a closed end portion integrally formed with the central portion, and an open end portion integrally formed with the central portion, wherein the open end portion of the second container comprises a mating portion; and the coupling device comprising a tubular central portion with first and second open ends and a porous insert extending horizontally across the coupling device, wherein each open end of the coupling device comprises a mating portion, wherein the porous insert has a pore size of 40 to 500 μm. The coupling device further includes a port extending radially outward from the tubular central portion, wherein one mating portion of the coupling device is attached to the mating portion of the first container and the other mating portion of the coupling device is attached to the mating portion of the second container. The port can include a porous insert.

The first and second containers can be pre-assembled, wherein an interior space defined by the first container and the second container is at least partially under vacuum.

This document also features a container that includes an elongated cylindrical central portion defining an internal lumen; a first end portion integrally formed with the central portion; a second open end portion integrally formed with the central portion; a shaft disposed vertically in the internal lumen; and a plurality of arms disposed along a length of the shaft and extending in a substantially radial direction from the shaft into the internal lumen. The plurality of arms can be of different shapes or sizes. The container further can include a removable lid that attaches to the second open end portion, wherein the shaft is affixed to the removable lid. The shaft can be rotatably affixed to the container. The shaft can be moveable within the lumen of the container.

In another aspect, this document features a kit that includes any of the containers or container assemblies described herein. The kit further one or more cell separation reagents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, Genbank® Accession Nos, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF DRAWINGS

FIG. 1 is a top view of a removable rotating apparatus that may be inserted into an automated tissue processing unit.

FIG. 2 is a side view of a removable rotating apparatus that may be inserted into an automated tissue processing unit.

FIG. 3 is a graph illustrating the effect of extruding the tissue sample prior to centrifugation.

FIG. 4 is a graph illustrating the additive effect of centrifugation of a tissue sample.

FIG. 5 illustrates the effect of an amount of time of centrifugation of the tissue sample.

FIG. 6 illustrates the effect of acceleration by centrifugal force on a tissue sample.

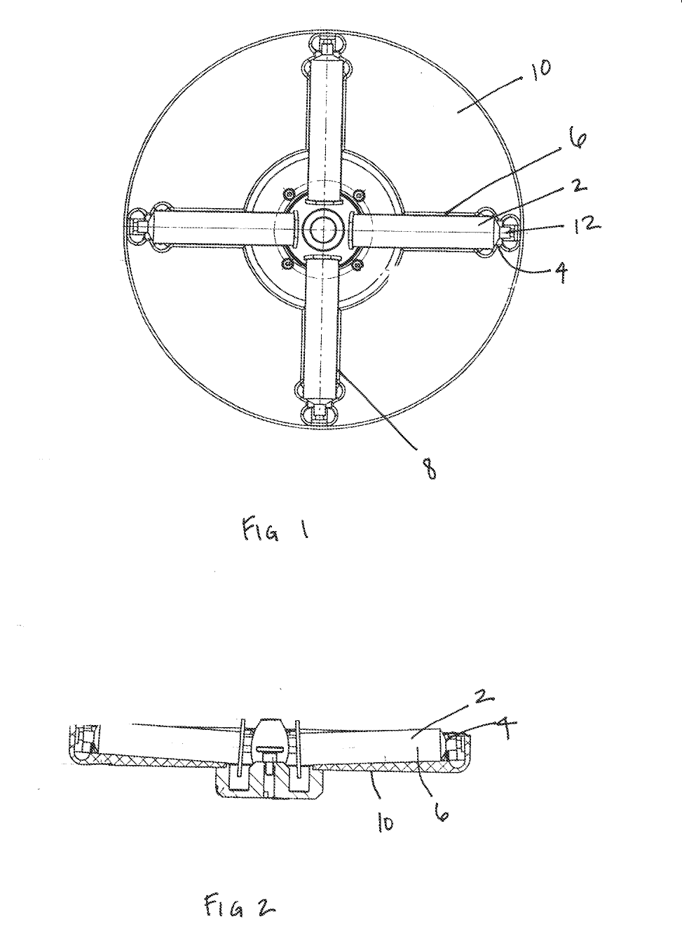

It will be appreciated that the removable rotating apparatus illustrated in FIGS. 1-2 are not to scale or proportion and that certain features of it may be exaggerated or distorted for illustrative purposes.

Like reference symbols in the various drawings indicate like elements.

### DETAILED DESCRIPTION

In general, this document is based on methods and apparatus for recovery of cells from tissues, including human and animal tissues. The methods and apparatus described herein are particularly useful for recovery of cells from adipose tissue obtained from, for example, liposuction (i.e., lipoaspirate), including suction assisted, vapor assisted, or ultrasound assisted liposuction. For instance, the methods and apparatus described herein can be used to isolate stem cells, progenitor cells, hematopoietic cells, or fully differentiated cells from adipose tissue.

The methods and apparatus described herein can be used on site to prepare cellular compositions for administration to a patient (e.g., autologous administration). For example, the methods and apparatus described herein can be used to recover regenerative cells from a patient that can be prepared for administration and then administered (e.g., injected or surgically implanted) back to the patient from which the cells were recovered. In some embodiments, the cells can be loaded into a delivery device such as a syringe, for injection into the recipient by, for example, subcutaneous, intravenous, intramuscular, or intraperitoneal techniques. For example, the cells can be injected into blood vessels for systemic or local delivery, into tissue (e.g., cardiac muscle or skeletal muscle), into the dermis (subcutaneous), into tissue space (e.g., pericardium or peritoneum), or other location. In some embodiments, one or more additives are added to the cells before administration. For example, the cells can be mixed with other cells, biologically active compounds, biologically inert compounds, demineralized bone, a matrix or other resorbable scaffold, one or more growth factors, or other additive that can enhance the delivery, efficacy, tolerability, or function of the cell population.

The methods and apparatus also can be used to prepare cellular compositions for growth studies, gene expression studies, differentiation studies, or other research purposes. In addition, the methods and apparatus described herein can be used to recover regenerative cell populations (e.g., stem cells) such that the cells can be banked, for example, by cryopreserving the cells with an appropriate medium. For further reference, see U.S. Patent Application Publication No. 20100285588-A1.

It has been discovered that subjecting a tissue sample housed in a tissue collection container to at least one round of acceleration by means of centrifugal force may produce a tissue sample concentrate having a regenerative platform therein. The tissue sample may be extracted from a species, such as but not limited to a human, a canine, a feline, an equine, a bovine, an ovine, or a porcine. The tissue sample may include, but is not limited to lipoaspirate, adipose tissue, and combinations thereof. The tissue sample may have a regenerative platform throughout the tissue sample; however, centrifugation causes the tissue sample to form a tissue sample concentrate, i.e. having a concentrated amount of the regenerative platform therein. For example, upon centrifugation of a tissue sample within a tissue collection container, there are three general layers that form, such as but not limited to, an aqueous pellet, a tissue sample concentrate having the regenerative platform, and a lipid layer. The tissue sample concentrate is generally located between the lipid layer and the aqueous pellet. After the extraction of the aqueous pellet, the tissue sample concentrate may then be easily extracted from the tissue collection container in a method that maintains a closed system for further use of the tissue sample concentrate.

The centrifugal force may be produced by an automated tissue processing unit having a removable rotating apparatus therein where the removable rotating apparatus is configured to rotate within the automated tissue processing unit. The automated tissue processing unit may include a temperature control device for controlling the temperature within the unit.

The automated tissue processing unit may also have a mechanism for identifying a particular removable rotating apparatus by at least one pre-determined specification of the removable rotating apparatus. In one embodiment, the removable rotating apparatus may have an attached RFID tag. The RFID tag may be scanned upon placement of the removable rotating apparatus into the automated tissue processing unit for identification by the automated tissue processing unit. In another embodiment, a specification of the removable rotating apparatus within the automated tissue processing unit may be measured and recorded, such as but not limited to the power demand associated with acceleration, weight, wind resistance, and combinations thereof. The measured specification may be stored as part of a software program and/or software package of the automated tissue processing unit that enables the automated tissue processing unit to identify the removable rotating apparatus by such specification data.

The tissue sample may be housed in a first tissue collection container adapted for the automated tissue processing unit. The tissue sample may have or include a suspension of tissue pieces in an aqueous fluid. In one non-limiting embodiment, the tissue sample may be extruded prior to placement of the tissue sample into the first tissue collection container. The tissue sample may be extruded between one and twenty times, or alternatively from about two times to about ten times through an orifice ranging in diameter from about 1 mm independently to about 4 mm, or alternatively from about 1.5 mm independently to about 3 mm. Once the extruded tissue sample has been subjected to a round of acceleration, the tissue sample concentrate that forms may have a higher concentration of the regenerative platform compared to an otherwise identical method absent the extrusion of the tissue sample. As used herein with respect to a range, “independently” means that any lower threshold may be used together with any upper threshold to give a suitable alternative range.

The tissue sample may be subjected to at least one round of centrifugation ranging from about 200×g independently to about 2000×g, or alternatively at least 400×g using the automated tissue processing unit. The centrifugation may occur for a time period ranging from about 3 minutes independently to about 60 minutes, or at least about 5 minutes. After the centrifugation, a tissue sample concentrate may form. The tissue sample concentrate includes, but is not limited to a regenerative platform therein where the regenerative platform may include an amount of regenerative cells and/or a regenerative matrix.

The tissue sample concentrate may then be transferred from the first tissue collection container into a second collection container by a closed system method. Such a closed system method may include but is not limited to a mechanism including, but not limited to a leur connector between the first collection container and the second collection container, a spike port, a needle, and combinations thereof. The closed system method of transfer keeps the tissue sample concentrate and regenerative platform from being contaminated by any additional pathogens external to the tissue sample and/or the tissue collection containers, such as but not limited to bacteria, viruses, and the like from entering into the tissue collection containers or the tissue sample concentrate. The closed system decreases the necessity for additional steps to be performed on the tissue sample concentrate prior to the administration of the tissue sample back into a subject. The administration of the tissue sample may be performed by a method, e.g. an injection. As used herein, the numeral notation of ‘first tissue sample container’ and ‘second tissue sample container’ denotes the usage order of the containers. The containers may be the same types of containers or different types of containers, e.g. a vial or centrifuge tube.

At least one protease may be added to the second tissue collection container having the transferred tissue sample concentrate, such as but not including collagenase, dispase, thermolysin, trypsin, and combinations thereof. The protease may be added to the second tissue collection container in an amount ranging from about 0.5 Wunsch units collagenase per ml independently to about 4.0 Wunsch units collagenase per ml, or alternatively from about 1.0 Wunsch units collagenase per ml independently to about 3.0 Wunsch units collagenase per ml.

The second tissue collection container may then be subjected to at least two rounds of acceleration where each round of acceleration is followed by a round of deceleration. Each round of acceleration and deceleration may occur until at a rate of at least about 10×g is obtained. Alternatively, the rate of acceleration and deceleration may occur at a rate ranging from about 10×g to independently about 400×g, or from about 20×g independently to about 40×g in another non-limiting embodiment. In one non-limiting embodiment, the number of rounds per minute of acceleration and deceleration may range from about 1 round per minute to about one round per five minutes, or alternatively at least about three rounds per minute. In another non-limiting embodiment, the tissue sample concentrate may be disaggregated after a number of rounds of acceleration and deceleration, or alternatively at least two rounds of acceleration and deceleration.

The intermittent rounds of acceleration followed by deceleration in the presence of a protease may disaggregate the regenerative platform of the tissue sample concentrate. After the acceleration and deceleration, the tissue sample concentrate may be filtered and washed to obtain a regenerative platform that may be administered back into a subject, e.g., an injectable regenerative platform. ‘Injectable’ is defined herein to mean that few or no additional steps must be performed for the regenerative platform to be injected into a subject.

After the filtering of the tissue sample concentrate, at least a portion of the injectable regenerative platform may be injected into an injection site whereby the injection alters an area near the injection site. ‘Alter’ is defined herein to include augmenting, repairing, reducing inflammation, reducing pain, and combinations thereof at the injection site.

FIG. 1 is a top view of a removable rotating apparatus 10 that may be inserted into an automated tissue processing unit (not shown). A tissue collection container 2 may be detachably inserted into the removable rotating apparatus 10 and held in place by a snappable locking mechanism 4. Here, the tissue collection container 2 snaps into the cavity 6. In one non-limiting embodiment, the tissue collection container 2 is customizable to snabbably fit within the cavity 6 and held in place by the snappable locking mechanism 4. The tissue collection container 2 is oriented so that the opening 12 of the tissue collection container 2 is farthest from the center. The formation of a tissue sample concentrate may form near the opening 12, and the tissue sample concentrate may be easily extracted from the tissue collection container 2 through the opening without additional contamination to the tissue sample concentrate.

The removable rotating apparatus 10 may have at least two cavities (shown here as 6, 8) or may have up to about eight cavities in another non-limiting embodiment. Each cavity 6, 8 may be in a horizontal orientation and may have a detachable mechanism for inserting a tissue collection container 2 within the cavity 6, 8. The detachable mechanism may be, but is not limited to a snapping mechanism, Velcro, and the like, and combinations thereof. In another non-limiting embodiment, the removable rotating apparatus 10 may have or include autoclavable materials, such that the removable rotating apparatus 10 is configured to be autoclavable.

FIG. 2 is a side view of a removable rotating apparatus 10 that may be inserted into an automated tissue processing unit (not shown). The tissue collection container 2 is shown within the cavity 6 and held in place by the snappable locking mechanism 4.

The invention will be further described with respect to the following Example which is not meant to limit the invention, but rather to further illustrate the various embodiments.

### EXAMPLES

### Example 1

Fresh canine omental adipose tissue was obtained from tissue discarded after spay surgery. Tissue was minced with sterile scissors and then equally divided (approximately 2 g/tube) into 50 mL sterile centrifuge tubes. Sterile lactated Ringer's containing a blend of bacterial collagenases I and II together with dispase was added and the tubes were then randomly assigned to incubation in a shaking incubator (60 rpm) or a heated tissue processing apparatus in a fixed rotor (TPA, 3 cycles per min of 1×g to 200×g to 1×g). Temperature was maintained between 37-40° C. and incubation/processing was conducted for 30 min.

After processing, the digested tissue slurry was passed through a 100 μm filter and the cell fraction was recovered from the filtrate by centrifugation at 400×g for 10 min in the TPA. Cell fractions were plated in 25 cm2 tissue culture flasks and grown for two days at 37° C. in DMEM/20% (v/v) fetal bovine serum (FBS) containing antibiotic and antimycotic. After culturing for two days, adherent cells were counted using a hemacytometer. Processing the tissue with the TPA resulted in a 2.6 fold higher than when digestion was performed in a shaking incubator.

### Example 2

Fresh human adipose lipoaspirate was obtained with patient informed consent from a patient undergoing elective lipoplasty. Tissue was drained using a sterile stainless steel strainer and then equally divided (approximately 10 g/tube) into 50 ml sterile centrifuge tubes with or without inserts fabricated from nylon mesh with 1 mm pore size. Sterile lactated Ringer's containing a blend of bacterial collagenases I and II together with dispase was added and the tubes then were randomly assigned to incubation in a shaking incubator (60 rpm) or a heated TPA in a swinging bucket rotor (3 cycles per min of 1×g to 200×g to 1×g). Temperature was maintained between 37-40 ° C. and incubation/processing was conducted for 30 min.

After processing, the digested tissue slurry was passed through a 100 μm filter and the cell fraction was recovered from the filtrate by centrifugation at 400×g for 10 min in the TPA. Cell fractions were plated in 25 cm² tissue culture flasks and grown for two days in DMEM/20% (v/v) FBS containing antibiotic and antimycotic. After culturing for two days, adherent cells were counted using a hemacytometer. Results indicate that cell yield obtained by processing in the TPA and using the cone insert is similar to cell yield obtained by processing in the incubator.

### Example 3

A lipoaspirate sample was obtained from a human patient undergoing elective lipoplasty. Lipoaspirate was transferred to a plurality of tissue collection containers having a 20 cc volume. Lipoaspirate contents of each of the tissue collection containers were extruded five times through a micro-emulsifying needle. The extruded lipoaspirate from each of the tissue collection containers was then transferred to a separate tissue collection container. The tissue collection containers were then placed into the cavities of a removable rotating apparatus within an automated tissue processing unit. The removable rotating apparatus allowed the tissue collection containers to maintain a horizontal position, while the automated tissue processing unit applied acceleration and centrifugal force to the contents of the tissue collection containers. The centrifugal force was applied for 30 minutes at a rate of about 400×g, about 700×g, about 1200×g, or about 2000×g for 30 minutes. The contents of two tissue collection containers were used for each rate of centrifugal force.

After the centrifugation, a tissue sample concentrate having a regenerative platform was separated from the remainder of the tissue sample within each tissue sample collection container. The volume of the layer of oil, a lipoaspirate fraction, and an aqueous fraction was removed and measured for each sample. The lipoaspirate layer was placed into a separate 50 cc conical centrifuge tube.

As a control sample, approximately 5 g of unprocessed, i.e., not extruded nor centrifuged, lipoaspirate was transferred to each of 2 tissue collection containers. The containers were weighed and the weight of the transferred lipoaspirate was recorded. Ringer's lactate that includes a collagenase enzyme and a dispase enzyme was added to the unprocessed lipoaspirate in an amount of 5 mL to each tissue collection container. The tissue collection containers were then placed into a shaking incubator at about 37 degrees C., and about 60 rpm for about 30 min to disaggregate the unprocessed lipoaspirate. The disaggregated tissue sample was then passed through a 100 μm steriflip filter. The filtered tissue was then centrifuged at a rate of about 600×g for about 10 minutes to recover regenerative cells. The recovered cells were placed in culture in minimum essential medium (MEM) with 20% (v/v) fetal bovine serum for 24 hours. The adherent cells were counted by a hemacytometer.

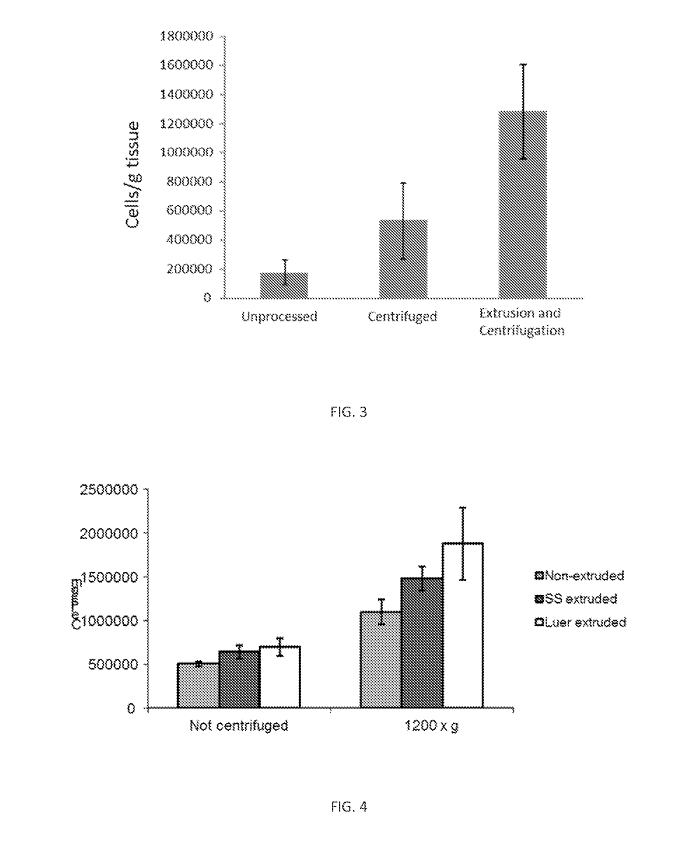

As illustrated in FIG. 3, extruding the tissue sample prior to centrifugation yields a greater number of cells per gram of tissue. FIG. 4 illustrates that centrifugation at 1200×g has an additive effect in terms of increasing the cell concentration within the tissue sample concentrate regardless of whether the tissue was extruded prior to centrifugation.

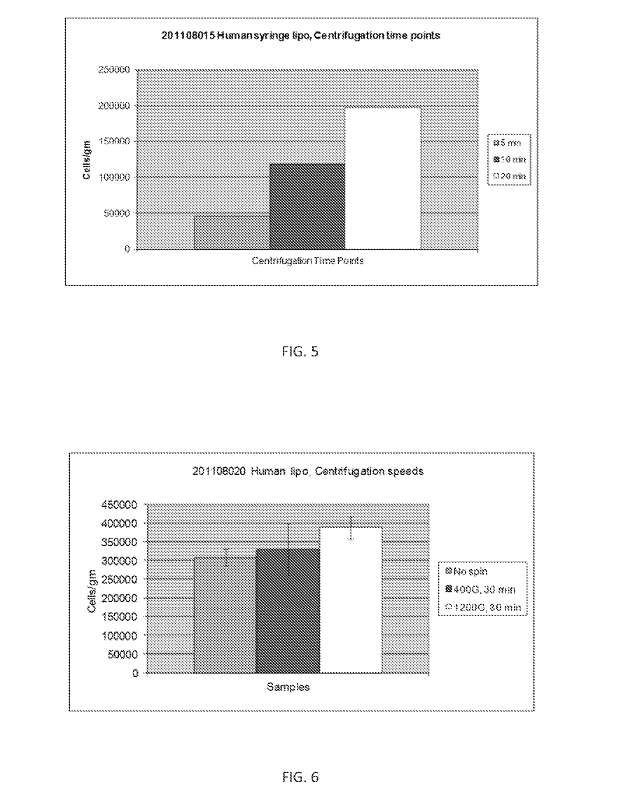

FIG. 5 illustrates that a longer amount of time for centrifugation of the tissue sample yields a higher number of cell per gram of tissue sample concentrate. FIG. 6 illustrates that a 1200×g rate of acceleration yields a higher concentration of cells per tissue sample concentrate when compared to a 400×g rate of acceleration or no centrifugation at all.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for recovering a regenerative platform from a tissue sample, said method comprising:
a) providing a tissue sample housed in a first tissue collection container adapted for an automated tissue processing unit, wherein the automated tissue processing unit comprises a removable rotating apparatus comprising at least two cavities, wherein each cavity is configured for detachably inserting a tissue collection container within the cavity wherein the tissue sample comprises a suspension of tissue pieces in an aqueous fluid; and
b) subjecting the tissue sample to at least one round of centrifugation of at least 400×g for at least about 5 minutes using the automated tissue processing unit thereby separating a tissue sample concentrate from the tissue sample, wherein the tissue sample concentrate comprises a regenerative platform.

2-68. (canceled)
